(19) United States
(12) Patent Application Publication
(10) Pub. No.: US 2024/0374240 A1
(43) Pub. Date: Nov. 14, 2024

(54) METHODS AND SYSTEMS FOR TRANSIENT TISSUE TEMPERATURE MODULATION

(71) Applicants: University of Pittsburgh - Of the Commonwealth System of Higher Education, Pittsburgh, PA (US); North Carolina State University, Raleigh, NC (US); Blatek Industries, Inc., State College, PA (US)

(72) Inventors: Kim Kang, Pittsburgh, PA (US); Qiyang Chen, Pittsburgh, PA (US); Zhiyu Sheng, Pittsburgh, PA (US); Bohua Zhang, Raleigh, NC (US); Howuk Kim, Raleigh, NC (US); Huaiyu Wu, Raleigh, NC (US); Xiaoning Jiang, Raleigh, NC (US); Mengyue Chen, Raleigh, NC (US); Xuecang Geng, State College, PA (US)

(73) Assignees: University of Pittsburgh - Of the Commonwealth System of Higher Education, Pittsburgh, PA (US); North Carolina State University, Raleigh, NC (US); Blatek Industries, Inc., State College, PA (US)

(21) Appl. No.: 18/690,699

(22) PCT Filed: Sep. 9, 2022
(86) PCT No.: PCT/US2022/043081

§ 371 Date: Mar. 8, 2024

Related U.S. Application Data
(60) Provisional application No. 63/242,163, filed on Sep. 9, 2021.

(51) Int. Cl. A61B 8/08; A61B 8/00

(52) U.S. Cl. CPC A61B 8/485; A61B 8/4477

(57) ABSTRACT

Ultrasound systems and methods of use are provided for rapidly heating a target area of a subject. The ultrasound system can include at least one imaging transducer array for imaging the target area, and at least two spaced-apart heating transducer arrays for heating the target area. The heating transducer arrays can be separated by the imaging transducer array on an ultrasound probe.

### CROSS-REFERENCE TO RELATED APPLICATION

This application claims the benefit of U.S. Patent Application No. 63/242,163, filed Sep. 9, 2021, which is incorporated by reference herein in its entirety.

### ACKNOWLEDGMENT OF GOVERNMENT SUPPORT

This invention was made with government support under Grant No. HL152023 awarded by the National Institutes of Health. The government has certain rights in the invention.

### FIELD

This disclosure is related to methods and systems for ultrasound imaging and transient tissue temperature modulation.

### BACKGROUND

Ultrasound-induced thermal strain imaging (US-TSI) can identify lipids in atherosclerosis plaque by tracking echo shifts due to tissue-composition-dependent sound speed change when the tissue temperature is increased. Current systems and methods suffer from various shortcomings, including issues relating to efficient operation and safety of use, and improvements to these systems and method are desirable.

### BRIEF DESCRIPTION OF THE DRAWINGS

FIG. 1 illustrates a schematic of a human carotid artery model.

FIG. 2 illustrates a schematic of an exemplary US-TSI device.

FIG. 3 illustrates a schematic of an exemplary US-TSI device having two spaced-apart heating arrays and an imaging array.

FIG. 4 illustrates a schematic of an exemplary heating array.

FIG. 5 illustrates a schematic of an exemplary US-TSI device.

FIG. 6 illustrates a bottom view of the exemplary US-TSI device shown in FIG. 5.

FIG. 7 illustrates the exemplary US-TSI device shown in FIG. 5 with a flexible, acoustically conductive membrane.

FIG. 8A illustrates the acoustic pressure field of a single heating array in the lateral-axial plane (i.e., the xz plane) and FIG. 8B illustrates the acoustic pressure field of a single heating array the elevational-axial plane (i.e., the yz plane).

FIG. 9A illustrates the acoustic pressure field of a dual heating array in the lateral-axial plane (i.e., the xz plane) and FIG. 9B illustrates the acoustic pressure field of a dual heating array the elevational-axial plane (i.e., the yz plane).

FIG. 10 depicts temperature distribution in a region of interest over time for a carotid artery model.

FIG. 11 represents a temporal curve of the temperature response at the focal area where the maximum temperature was achieved.

### DETAILED DESCRIPTION

The following is a detailed description of various embodiments of the present invention. The aforementioned drawings are referenced to serve as some, not all, of the visual embodiments of the invention. It should be understood that all description and drawings are to be considered exemplification of the invention and is not intended to limit the invention to the specific embodiments described and illustrated below.

The systems and methods described herein, and individual components thereof, should not be construed as being limited to the particular uses or systems described herein in any way. Instead, this disclosure is directed toward all novel and non-obvious features and aspects of the various disclosed embodiments, alone and in various combinations and subcombinations with one another. For example, any features or aspects of the disclosed embodiments can be used in various combinations and subcombinations with one another, as will be recognized by an ordinarily skilled artisan in the relevant field(s) in view of the information disclosed herein. In addition, the disclosed systems, methods, and components thereof are not limited to any specific aspect or feature or combinations thereof, nor do the disclosed things and methods require that any one or more specific advantages be present or problems be solved.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed things and methods can be used in conjunction with other things and methods. Additionally, the description sometimes uses terms like “provide,” “produce,” “determine,” and “select” to describe the disclosed methods. These terms are high-level descriptions of the actual operations that are performed. The actual operations that correspond to these terms will vary depending on the particular implementation and are readily discernible by one of ordinary skill in the art having the benefit of this disclosure.

As used in this application the singular forms “a,” “an,” and “the” include the plural forms unless the context clearly dictates otherwise. Additionally, the term “includes” means “comprises.” Further, the term “coupled” or “secured” encompasses mechanical and chemical couplings, as well as other practical ways of coupling or linking items together, and does not exclude the presence of intermediate elements between the coupled items unless otherwise indicated, such as by referring to elements, or surfaces thereof, being “directly” coupled or secured. Furthermore, as used herein, the term “and/or” means any one item or combination of items in the phrase. As used herein, the term “exemplary” means serving as a non-limiting example, instance, or illustration. As used herein, the terms “e.g.,” and “for example,” introduce a list of one or more non-limiting embodiments, examples, instances, and/or illustrations.

Certain terms may be used such as “up,” “down,” “upper,” “lower,” and the like. These terms are used, where applicable, to provide some clarity of description when dealing with relative relationships. But, these terms are not intended to imply absolute relationships, positions, and/or orientations. For example, for convenience, some orientations are described with reference to the ultrasound device (or probe) being in a vertical position with the imaging and heating arrays facing downward.

As used herein, the terms “ultrasound transducer” and “transducer” have their ordinary meanings as understood by those skilled in the art of ultrasound imaging technologies. Any suitable transducer may be used. For example, in some embodiments, an ultrasound transducer may comprise a piezoelectric device. The transducers described herein are configured in arrays of multiple individual transducer elements. As used herein, the terms “transducer array” or “array” refers to a collection of transducer elements attached to a common support structure.

Unless explained otherwise, all technical and scientific terms used herein have the same meaning as commonly understood to one of ordinary skill in the art to which this disclosure belongs.

One challenge of current US-TSI for human subjects is the demand for a very fast temperature rise in a relatively large volume with appropriate acoustic power under the FDA safety limit to cover a major artery, such as the carotid artery, and to avoid any physiologic motion artifacts.

As described herein, in some embodiments the disclosed ultrasound devices and methods of use can increase tissue temperature within a fraction of second and measure the temperature change remotely and noninvasively. The ultrasound energy can be delivered transiently to only the target location using, for example, a high power ultrasound heating array and the temperature can be measured using a co-localized highly sensitive ultrasound imaging array.

In some embodiments, the device can be used for imaging purposes including but not limited to identifying lipids in blood vessel walls, skeletal muscles, kidney, and liver. The device can also be used for therapy and monitoring such as hyperthermia for cancer treatment and pain relief, drug delivery, tissue ablation, as well as body lipid profile and lipid injection surgery.

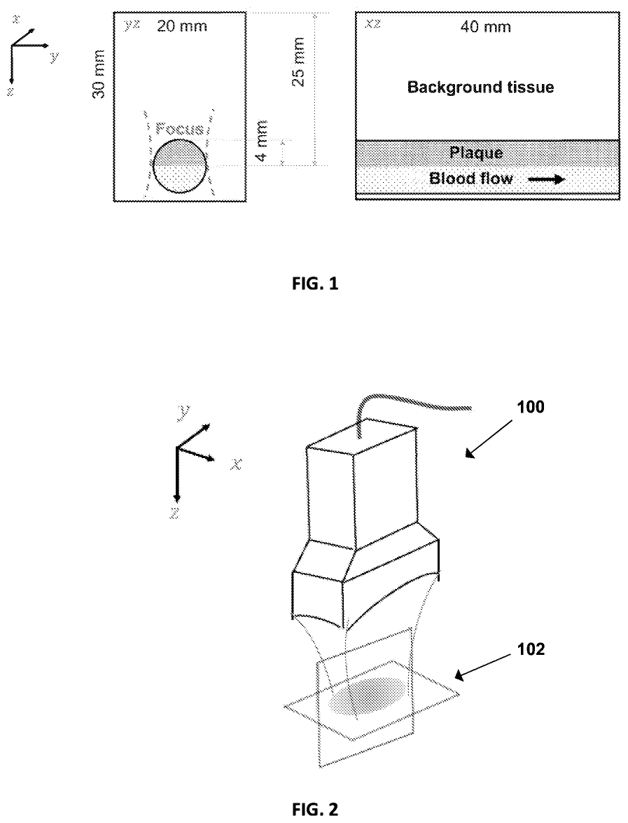

FIG. 1 provides a schematic of a human carotid artery model, which is an exemplary target area for the US-TSI devices described herein. A human carotid artery typically has a diameter that ranges from about 4 mm to 7 mm, and has a ⅛^{th} cardiac cycle (i.e., a period with less than 1 mm displacement of artery motion) ranges from 75 ms to 125 ms. In some embodiments, it is desirable to raise the temperature of a volume of a target area by a desired amount (e.g., 2° C.) in less than the ⅛^{th} cardiac cycle, such as less than 75 ms, or more preferably, in less than 50 ms, so that the main region of interest can be covered and the echo shift can be tracked with reduced and/or minimal physiological motion artifacts.

In one embodiment, the systems and methods can provide a heating transducer array capable of inducing a heating volume of 2×10×10 mm³ (in the x, y, and z directions) in a more effective manner (e.g., about a 2° C. temperature rise within 50 ms in some embodiments). FIG. 1 illustrates an ultrasound thermal strain imaging (US-TSI) device 100 that is heating and imaging a target area 102 (e.g., a target volume of a carotid artery).

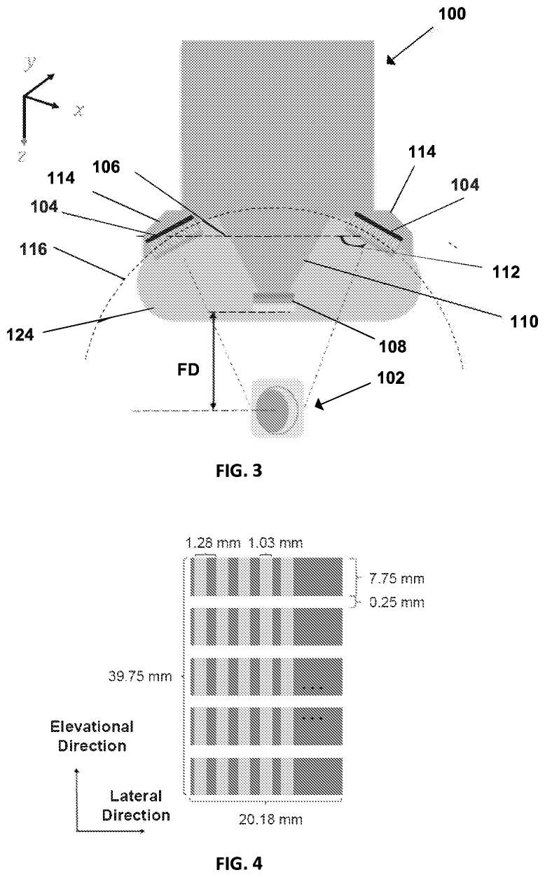

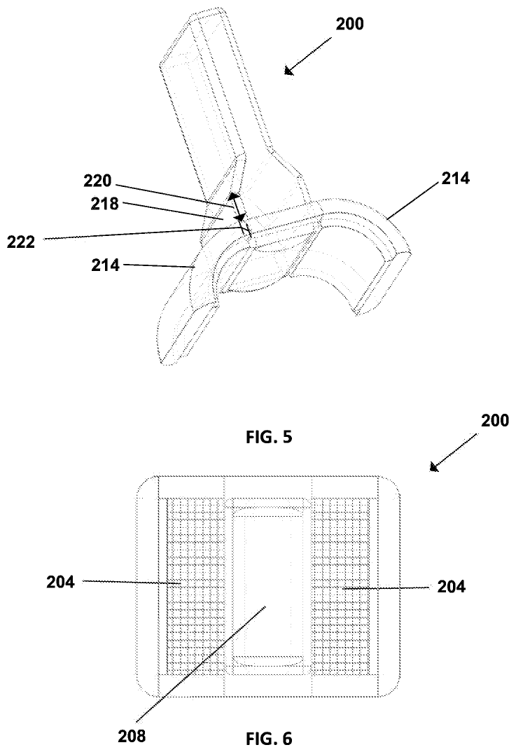

As shown in FIG. 3, the US-TSI device 100 comprises two heating arrays 104 positioned on opposite sides of an imaging array 108. Heating arrays 104 can be spaced apart along a horizontal line (axis) 106, which is generally parallel with a bottom surface of the US-TSI device 100. As shown in FIG. 3, the horizontal line 106 can be positioned to intersect a centerline of the heating arrays in the illustrated direction.

The two heating arrays 104 can be symmetrical arrays. The focus depths of the heating arrays can be set based on a curvature radius 116 of the US-TSI device 100 and other operational goals. In some embodiments, a focal depth FD of the imaging array can be between 20-30 mm (e.g., about 25 mm) and the focal depths of each heating array (shown in dashed lines in FIG. 3) can be between 40-60 mm (e.g., about 50 mm). Accordingly, in some embodiments, a ratio of the focal depths of the heating array to the focal depth of the imaging array can be 3:1 to 4:3.

The focal depth of the heating arrays can also be described in terms of the lateral focus (i.e., the focal depth in the xz plane) and the elevation focus (i.e., the focal depth in the yz plane). In some embodiments, the lateral focus can vary from 30-50 mm (e.g., about 40 mm) and the elevation focus can vary about 50-70 mm (e.g., about 60 mm).

The heating arrays 104 can be coupled to arms 114 that laterally extend from a main body of the US-TSI device 100 so that they are spaced apart from one another by a centrally positioned imaging array 108. The imaging array 108 can be vertically spaced from the heating arrays 104 by a centrally-extending portion 110, which extends vertically from the main body. In this manner, the imaging array 108 can have a shorter focus depth relative to the focus depth of the heating arrays.

The heating arrays 104 can be positioned at an angle relative to the orientation of the imaging array. For example, as shown in FIG. 3, an angle 112 can be formed between line 106 and the heating arrays 104. In some embodiments, angle 112 can range from 135 to 175 degrees, or, in other embodiments, from 145 to 165 degrees, or, in still other embodiments, from 150 to 160 degrees. In addition, as discussed in more detail below, in some embodiments, the heating arrays 104 can be movable so that the location and position of the heating arrays 104, including the angle 112, can vary depending on the application.

In some embodiments, the active material for the array elements of the heating arrays 104 can comprise PZT-4, and the materials of the matching layers and backing layers can be Al2O3 epoxy and air, respectively. In some embodiments, for dual-focus beamforming, the heating arrays 104 can comprise 1.5D arrays with different focal depth in the xz and yz planes. A membrane 124 can be provided to extend over some or all of the heating arrays and/or the imaging array.

The heating arrays preferably have greater number of elements in the x-direction than sections in the y-direction. For example, each heating array can have 5 sections along the y-direction, and each section can have 16 elements along the x-direction (i.e., 5×16 1.5D array). In an exemplary embodiment, additional details of the heating array can be as follows: Center frequency: 3.5 MHz, Number of elements: 5×16, Pitch along the x-direction: 1.28 mm, Pitch along the y-direction: 8 mm, and Kerf: 0.25 mm. FIG. 4 illustrates an exemplary structure of a 5×16 1.5 D array.

Each heating array can also include an acoustic lens to provided improved focus on the target area. Both heating arrays can use the same type of acoustic lens; alternatively, it may be desirable to use different acoustic lenses. In some embodiments, the acoustic lens can be a lens layer that received on the transducer array and/or connected to the common support structure (e.g., the arms).

FIG. 5 illustrates another exemplary US-TSI device 200. This device is similar to that disclosed in FIG. 3; however, the arms 214 which support the heating arrays are moveable relative to the main body of the US-TSI device 200. As shown in FIG. 5, the arms 214 can move up and down along supporting portion 218 of the main body in the directions indicated by arrow 220. In addition to being height adjustable, arms 214 can be angle adjustable. A pivoting member 222 (e.g., an adjustable joint/hinge) permits angle adjustments of the arms relative to the main body. Both adjustment members (height and angle) can have one or more locking members that secure the arms 214 in a desired position after movement/adjustment.

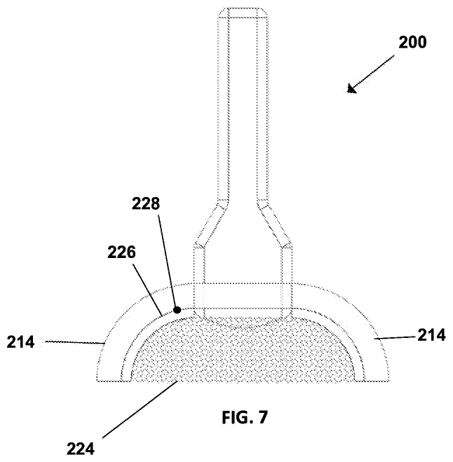

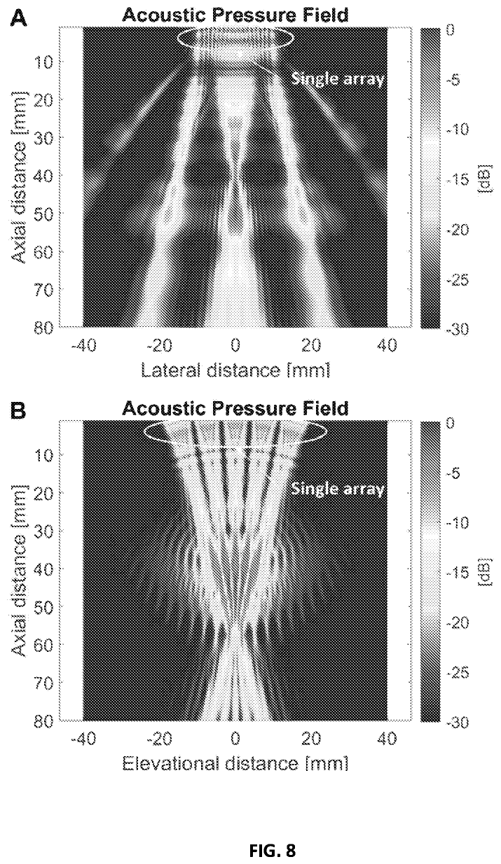

FIG. 6 illustrates a bottom view of the US-TSI device 200 shown in FIG. 5. As shown in FIG. 6, the two heating arrays 204 are lateral spaced from one another by a central imaging array 208. As shown in FIG. 7 (and in FIG. 3), membrane 224 can be provided. Membrane 224 can be a flexible, acoustically conductive membrane that is suitable for contact with the skin of a patient. Arms 214 can also have an internal cooling member 226, such as an embedded water-cooling configuration. The internal cooling member 226 can reduce heat generation and reduce the risk of the US-TSI device overheating and improve comfort of use. A safety temperature sensor and switch 228 can also be provided to further reduce risk. The safety sensor and switch can be configured to disable and/or turn off the heating arrays and/or US-TSI device when it is determined that a predetermined temperature is exceeded.

Acoustic simulation was performed to determine the acoustic pressure field induced by the US heating arrays using Field II. The elevation focus (i.e., the focal depth in the yz plane) was set based on curvature radius, and the lateral focus (i.e., the focal depth in the xz plane) was set based on phase delay. After obtaining the ultrasound beam pattern of a single heating array, we generated the beam pattern of dual heating arrays by rotating and moving the beam profile of two single heating arrays using MATLAB (R2020a, MathWorks, MA, USA). Various configurations of dual heating arrays were tested to ensure the focal area is right on target, as well as to ensure that the ultrasound wave propagation of the heating arrays is not affected by the imaging array. Corresponding simulation parameters used in Field II in an exemplary embodiment include sound speed: 1540 m/s, density: 1000 kg/m³, focal depth (lateral focus): 40 mm, and focal depth (elevation focus): 60 mm.

Based on the ultrasound beam profile of the dual heating arrays, finite difference method (FDM)-elicited thermal simulation on a human carotid artery model, as shown in FIG. 1, to determine desired amounts of acoustic pressure for the desired heating volume. The convection-diffusion equation was used to solve for the temperature field in a transient period. Acoustic pressure was transmitted as a continuous wave (i.e., 100% duty cycle) until 50 ms, and was turned off after that. Neumann boundary condition (NBC) were used for the top surface to simulate air-body boundary and Dirichlet boundary conditions (DBC) was used for all the other surfaces to mimic body condition. The initial condition was normal human body temperature (i.e., 36.7° C.). Additional simulation parameters related to the thermal properties are listed in the table below.

| | Tissue | | Tissue | | Tissue | |
|---|---|---|---|---|---|---|
| | Plaque | | Plaque | | Plaque | |
| | Blood | | Blood | | Blood | |
| Thermal | 0.476 | W/(m · K) | 0.484 | W/(m · K) | 0.549 | W/(m · K) |
| conductivity | | | | | | |
| Density | 1075 | kg/m3 | 920 | kg/m3 | 1050 | kg/m3 |
| Heat Capacity | 3490 | J/(kg · K) | 4080 | J/(kg · K) | 4390 | J/(kg · K) |
| Flow Speed | 0 | | 0 | | 0.4 | m/s |
| Absorption | 9 | Np/m | 7 | Np/m | 1.5 | Np/m |

coefficient

FIGS. 8A and 8B illustrate the acoustic pressure field of a single heating array in the lateral-axial plane (i.e., the xz plane) and in the elevational-axial plane (i.e., the yz plane), respectively. The results demonstrated that-6 dB focal area (i.e., the FWHM of acoustic pressure) is about 1.9×10.0×25.4 mm3 (in the x-, y-, and z-directions) and that the dual-focus beamforming approach using a 1.5D array can generate a wide US beam pattern (i.e., >10 mm) in the yz plane while confining the side lobes in the xz plane

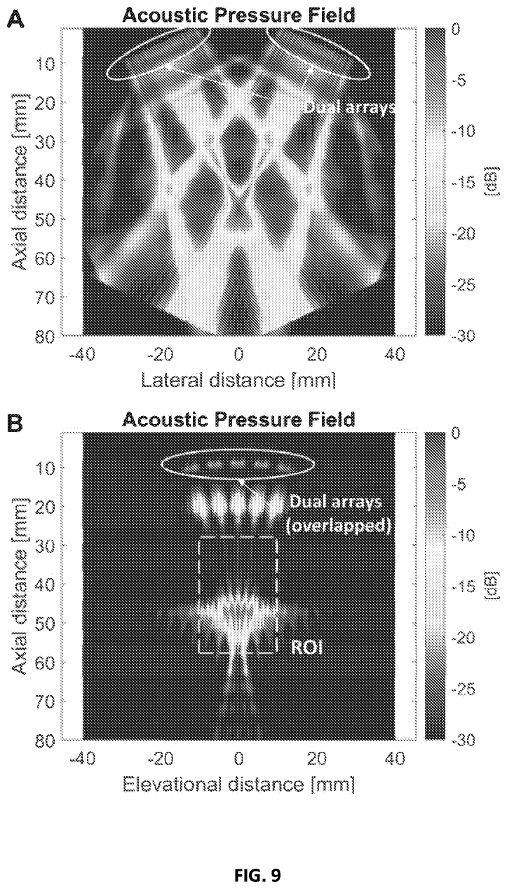

FIG. 9A illustrates the acoustic pressure field of a dual heating array in the lateral-axial plane (i.e., the xz plane) and FIG. 9B illustrates the acoustic pressure field of a dual heating array the elevational-axial plane (i.e., the yz plane). As shown in FIG. 9A, the distance between the two heating arrays was about 40 mm (as measured from a center of each array), and the rotation angle was 25 degrees from horizontal (or 155 degrees as shown in FIG. 3). In some embodiments, the centers of the spaced-apart heating arrays can range from 20-60 mm, or from 30-50 mm. This configuration of dual heating arrays generated a desirable ultrasound beam pattern. The focal area of a single array was overlapped with the other very well, reducing the influence upon surrounding tissue.

The sound pressure level (SPL) at an axial distance of 26 mm is also relatively low (i.e., <−25 dB), indicating that this location is suitable for placing imaging array and not influencing ultrasound wave propagation. It should be noted that the −6 dB beamwidth of dual heating arrays in the yz plane is decreased to about 8 mm, which was due to the fact that the maximum acoustic pressure is increased and the ultrasound beam pattern is rotated. Given that the maximum human carotid artery diameter is about 7 mm, the dual heating arrays provide sufficient beamwidth.

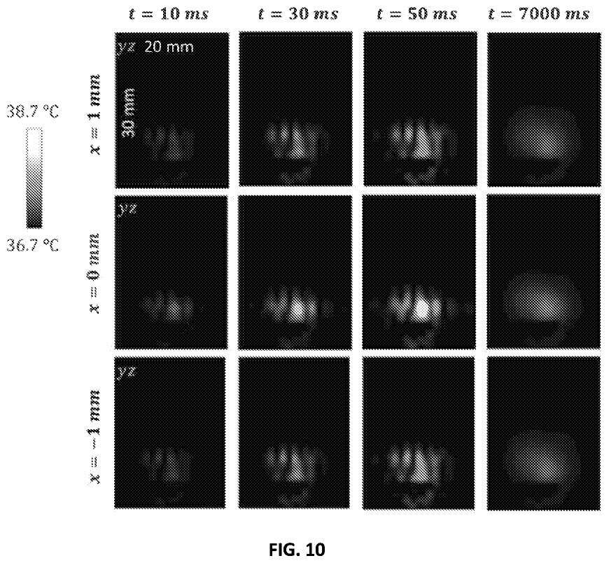

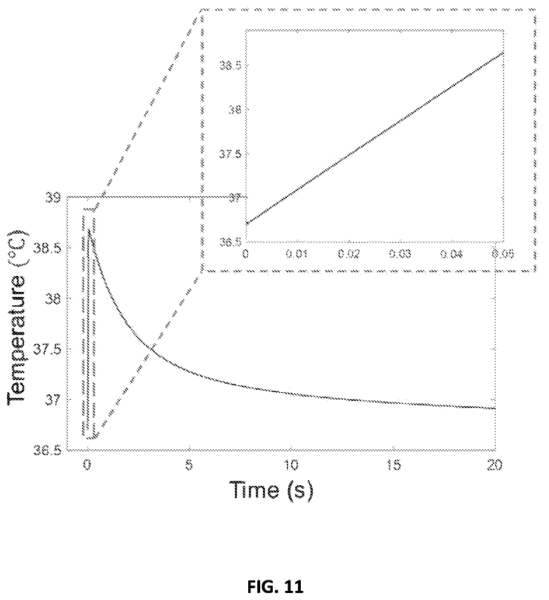

A finite-difference model was implemented to approximate the transient-to-steady state of the diffusion-convection process. The predicted temperature responses in the carotid artery model are illustrated in FIGS. 10A and 10B. Specifically, FIG. 10A shows the temperature distribution in the region of interest (ROI) to the designed heating beam for various temporal points and spatial slices. The ROI is aligned with the carotid artery model. The results showed that the heated volume (8.5 mm beamwidth) sufficient heats the desired volume. FIG. 10B represents the temporal curve of the temperature response at the location where the maximum temperature was achieved, the location is around the focal area of the ultrasound beam. It can be observed that the temperate rise is 2.1° C. within 50 ms when the RMS of the applied acoustic pressure is 4.5 MPa.

Overall, the background tissue and plaque were heated with a temperature rise of about 2° C. by the transmitted pressure field while the blood flow maintains the constant body temperature. Thermal diffusion was slow compared to the ˜50 ms time scale, and the required volume where the temperature rise needs to be controlled can be generally determined by the pattern of the ultrasound beam.

The exemplary ultrasound systems disclosed herein comprising at least two spaced-apart heating arrays and at least one imaging array. In some embodiments, the system can include one or more of a cooling system, an ultrasound temperature sensor, an ultrasound heating sensor (with an acoustic lens), a flexible membrane that is acoustically conductive positioned adjacent transducer arrays, and/or a safety temperature sensor and switch. As described herein, the heating transducers can be mounted on a body portion of the imaging transducer. In some embodiments, the heating transducers can be height/angle adjustable along the body portion. The heating transducers, for example, can be pivotable about and along a joint between the heating transducers and imaging array.

The systems and method described herein provide a US-TSI device that can more effectively increase the tissue temperature within the fraction of second in the area aimed by an aligned imaging ultrasonic sensor array device. In addition, in certain embodiments, the system can include: an uniquely angled heating array device along with multi-focused beam allows an effective temperature rise in an extended area and depth, an acoustic lenses for an effective focus, a joint between heating array(s) and imaging array allows the height and angle adjustment, an uniquely designed cooling system integrated into the ultrasound array device that allows for high power delivery without damaging the ultrasonic sensor elements and the skin surface of the subject. In some embodiments, using these systems, a very high-power deposit during a short period of time within a fraction of second can allow an effective temperature increase with minimum required power, without losing the heat by blood flow that carries out the heat by perfusion, conduction, and convection.

In some embodiments, the dual heating arrays are configured to heat a target area of the subject (e.g., a carotid artery) to increase the temperature between about 1-3° C., or about 1.5° C.-2.5° C., such as 2° C., in a very short time period, preferably between 25 ms-100 ms, between 25 ms-75 ms, between 25 ms-50 ms, or within 75 ms or, alternatively, within 50 ms.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. An ultrasound probe for rapidly heating a target area of a subject, the probe comprising:
at least one imaging transducer array for imaging the target area; and
a first heating transducer array for heating the target area and a second heating transducer array for heating the target area, the first heating transducer array and the second heating transducer array being spaced apart from one another,
wherein the at least one imaging transducer array and the first and second heating transducer arrays are aligned to have a common focus area.

2. The ultrasound probe of claim 1, wherein the first and second heating transducer arrays are separated by the at least one imaging transducer array.

3. The ultrasound probe of claim 1, wherein the at least one imaging transducer array is centered between the first and second heating transducer arrays.

4. The ultrasound probe of claim 1, further comprising:
a main body, a first arm that extend from the main body and supports the first heating transducer, and a second arm that extends from the main body and supports the second heating transducer.

5. The ultrasound probe of claim 4, further comprising:
a centrally-extending member that extends from the main body and supports the at least one imaging transducer array at a position below that of the positions of the first and second heating transducer arrays.

6. The ultrasound probe of claim 4, wherein the first and second heating transducer arrays are height adjustable along the main body of the at least one imaging transducer.

7. The ultrasound probe of claim 6, wherein the first and second heating transducer arrays are movably coupled to a side of the main body.

8. The ultrasound probe of claim 4, wherein the first heating transducer is positioned at a first angle relative to a horizontal axis, and the second heating transducer is positioned at a second angle relative to a horizontal axis.

9. The ultrasound probe of claim 8, wherein the first angle and the second angle are between 135 degrees and 175 degrees, between 145 degrees and 165 degrees, or between 150 and 160 degrees.

10. The ultrasound probe of claim 4, further comprising an integrated cooling system that extends into the first and second arms.

11. The ultrasound probe of claim 1, further comprising a first acoustic lens received on the first heating transducer array and a second acoustic lens received on the second heating transducer array.

12. The ultrasound probe of claim 1, further comprising at least one acoustically conductive flexible membrane positioned adjacent the at least one imaging transducer array and the first and second heating transducer arrays.

13. A method of performing ultrasound-induced thermal strain imaging of a target area of a living human subject in vivo, the method comprising:
providing an ultrasound probe having at least one imaging transducer array for imaging the target area and at least two spaced-apart heating transducer arrays for heating the target area;
focusing the at least two spaced-apart heating transducer arrays on the target area and heating the target area to increase a first temperature of the target area to a second temperature of the target area within 75 ms;
imaging the heated target area with the at least one imaging transducer array; and
determining an echo shift due to composition-dependent sound speed changes at the target area at the first temperature and at second temperature,
wherein the second temperature is between 1° C. and 3° C. higher than the first temperature.

14. The method of claim 13, wherein the second temperature is between 1.5° C. and 2.5° C. higher than the first temperature, and the first temperature is increased to the second temperature within 50 ms.

15. The method of claim 13, wherein the strain imaging probe of claim 1, wherein the at least two spaced-apart heating transducer arrays comprise first and second heating transducer arrays that are separated by the at least one imaging transducer array, wherein the first and second heating transducer arrays are each mounted on an arm that extends laterally from a main body of the ultrasound probe.

16. The method of claim 15, wherein the first and second heating transducer arrays are movably mounted and the method further comprises:
adjusting a position of the first and second heating transducer arrays.

17. The method of claim 16, wherein adjusting the position of the first and second heating transducer arrays comprises changing an angle of at least one of the first and second heating arrays relative to a horizontal axis.

18. The method of claim 16, wherein adjusting the position of the first and second heating transducer arrays comprises changing a height of the at least one of the first and second heating arrays relative to a lower surface of the ultrasound probe.

19. The method of claim 15, further comprising:
cooling one or more portions of the ultrasound probe while the target area is heated.

20. The method of claim 19, wherein the cooling of the one or more portions comprises circulating water of a cooling system within the arms on which the first and second heating transducer arrays are mounted.

21. (canceled)

22. (canceled)
